(19) United States
(12) Patent Application Publication
(10) Pub. No.: US 2010/0136029 A1
(43) Pub. Date: Jun. 3, 2010

(54) Use of Tri-Substituted Glycerol Compounds for the Treatment of Hematological Malignancies

(75) Inventors: Axel Rolf Zander, Hamburg (DE); Francis Ayuk Ayuketang, Hamburg (DE); Wolfgang Richter, Munich (DE); Lutz Weber, Germering (DE)

Correspondence Address: K&L Gates LLP, P.O. Box 1135, CHICAGO, IL, 60690, US

(73) Assignees: UNIVERSITATSKLINIKUM HAMBURG-EPPENDORF, Hamburg (DE); ALPHAPTOSE GMBH, Hamburg (DE)

(21) Appl. No.: 12/520,354

(22) PCT Filed: Nov. 9, 2007
(86) PCT No.: PCT/EP2007/062178

§ 371 Date: Feb. 3, 2010

(30) Foreign Application Priority Data
Dec. 20, 2006 (US) 60875963

(51) Int. Cl. A61K 31/661; A61P 35/00; A61P 35/02; A61K 33/24; A61K 31/704; A61K 39/395; C12Q 1/68

(52) U.S. Cl. 424/173.1; 514/114; 424/649; 514/34; 435/6

(57) ABSTRACT

The present invention relates to the use of a tri-substituted glycerol compound for the manufacture of a medicament for the treatment of hematological malignancies. The invention also refers to a kit-of-parts comprising such a medicament. Finally, the invention also relates to a corresponding method for the treatment of such conditions as well as to an in vitro method for determining the susceptibility of such malignant cells to a medicament as defined in the invention.

The present invention relates to the use of a tri-substituted glycerol compound for the manufacture of a medicament for the treatment of hematological malignancies. The invention also refers to a kit-of-parts comprising such a medicament. Finally, the invention also relates to a corresponding method for the treatment of such conditions as well as to an in vitro method for determining the susceptibility of such malignant cells to a medicament as defined in the invention.

Hematological malignancies such as leukemia and lymphoma are the types of cancer that affect blood, bone marrow and lymph nodes. Chromosomal translocations strongly influence the etiology of these diseases, while this is uncommon in solid tumors. This leads to a different approach in diagnosis and treatment of hematological malignancies (see, e.g., Dewald, G. W. et al. (1995) Semin. Oncol. 22, 341-354; Grimwade, D. et al. (1998) Blood 92, 2322-2333; Jaffe, E. S. et al. (2001) Tumors of hematopoietic and lymphoid tissue. International Agency for Research on Cancer, IARC Press, Lyon, France; Grimwade, D. et al. (2004) Ann. Hematol. 83, Suppl. 1, S45-S48).

Many patients with disseminated hematological malignancies are treated with chemotherapy (optionally in combination with irradiation) at some point during their illness. While response rates are generally high in some malignancies, such as lymphoid malignancies, only a subset of patients will be cured. Even amongst the lymphoid malignancies some (e.g. chronic lymphocytic leukemia (CLL), stage III/IV follicular small cleaved cell lymphoma, stage III/IV marginal zone lymphoma, stage III/IV mantle cell lymphoma, and multiple myeloma) are currently incurable with standard dose chemotherapy.

However, the currently available non-surgical therapeutic options are associated with adverse drug reactions, some of which are severe, which represents a limiting factor especially for chemotherapeutic approaches for an increasingly aging population with multiple pathologies.

One problem to be addressed in the treatment of such malignancies that recurrence of these tumors is unavoidable even with, or despite, the use of aggressive therapeutic regimen. Long-term therapy with chemotherapeutics is not possible because of the toxicity of these substances, and repeated use is not worthwhile because the response rates then approach zero. The limit for radiation exposure is likewise reached relatively quickly, if it is intended to treat even the primary tumor effectively. Thus, the situation is still such that after completion of the first therapeutic regimen it is necessary to “wait” until there has been renewed occurrence.

Presently, the chemotherapies for the treatment of hematological malignancies involve the administration of one or more anti-neoplastic agents (i.e. cytostatic and/or cytotoxic chemotherapeutics) such as alkylating agents, plant alkaloids, antimetabolites, antibiotics, inhibitors of topoisomerase I and/or II, inhibitors of tyrosine kinases, proteosome inhibitors, and steroids (cf., for example, Alexanian, R. et al. (1969) JAMA 208, 1680-1685; Rai, K. R. et al. (1981) Blood 58, 1203-1212; Alexanian, R. et al. (1990) Am. J. Hematol. 33, 86-89; Bassan, R. et al., 1992, Leukemia 6 (suppl. 2), 186-190; Keating, M. J. (1993) Chemotherapy of chronic lymphocytic leukemia, in: Cheson, B. D. (ed.) Chronic Lymphocytic Leukemia. Scientific Advances and Clinical Developments. Marcel Dekker, New York, N.Y., p. 297 ff.; Boulard, F. et al. (1993) Cancer Invest. 11, 534-553; Smith, M. et al. (1996) J. Clin. Oncol. 14, 18-24; Noordijk E. et al. (1997) Int. J. Radiat. Oncol. Biol. Phys. 39, 173; Miller, T. P. et al. (1998) N. Eng. J. Med. 339, 21-26; Drucker, B. J. (2001) N. Engl. J. Med. 344, 1031-1037; Richardson, P. G. et al. (2002) ASCO Annual Meeting Proceedings, 11a). All common anti-neoplastic agents have to be administered systematically, as they represent molecules also exerting nonselective effects on their physiological targets. However, this implies that such anti-neoplastic (cytostatic) agents, especially if applied over a long period of time, will have a harmful effect on the body's normal cells, i.e. its use is associated with significant adverse side effects mainly affecting the fast dividing body cells. Common side effects include inter alia genetic damage, nausea, vomiting, diarrhea, constipation, hair loss, immune suppression, myelo-suppression, anemia, hemorrhage, cytotoxicity, secondary neoplasms, and the like).

Thus, there is still a need either for alternative chemotherapeutics displaying less adverse side effects while preserving their pharmacological efficacy or for any compounds to be administered in combination with the above anti-neoplastic agents, wherein the adjunction results in a synergistic cytostatic and/or cytotoxic effect on malignant cells as compared to an individual application. Such adjunction would again allow administering a reduced dosage of anti-neoplastic agents to achieve the same therapeutic efficacy thus resulting in less severe adverse side effects.

Tri-substituted glycerol compounds belonging to the class of synthetic ether-linked alkyl-lysophospholipids might be candidate compounds for such an combinatorial approach.

Such synthetic ether-linked alkyl-lysophospholipids are known to have an anti-cancerogenic activity (reviewed, e.g., by Arthur, G., and Bittman, R. (1998) Biochim. Biophys. Acta 1390, 85-102; Jendrossek, V., and Handrick, R. (2003) Curr. Med. Chem. Anti-Canc. Agents 3, 343-353; Mollinedo, F. et al. (2004) Curr. Med. Chem. 11, 3163-3184). 1-O-octadecyl-2-O-methyl-glycero-3-phosphocholine (also referred to as ET-18-OCH3, AP-121 or edelfosine) is considered to be the prototype of these lipids. 1-O-octadecyl-2-O-methyl-glycero-3-phosphocholine represents a synthetic analogue of the platelet activating factor (PAF; 1-O-alkyl-2-acetyl-sn-glycero-3-phosphocholine), a potent phospholipid activator and mediator of many leukocyte functions, including platelet aggregation, inflammation, and anaphylaxis. Unlike most conventional chemotherapeutic drugs, these synthetic ether lipids do not directly target cellular DNA but rather affect the plasma membrane lipid composition and/or interfere with various signal transduction pathways. Thus, their mode of action does not depend on the presence of particular cellular receptors or is it cell cycle-dependent.

Cancer chemotherapy generally aims to slow the growth of, or destroy, cancer cells while avoiding collateral damage to surrounding cells and tissues. Consequently, the most effective anticancer agents are those that are able to selectively target cancer cells while leaving normal cells relatively unaffected. Synthetic ether-lipids have been shown to exert such an effect (cf., for example, Magistrelli, A. et al. (1995) Drug. Metab. Dispos. 23, 113-118). Several mechanisms of action have been proposed for the toxicity of ether-lipids towards cancer cells, including the cells' lack of alkyl cleavage enzymes. The resultant inability to hydrolyze the ether-lipids leads to their intracellular accumulation and to consequent damage to cell membrane lipid organization. Other potential mechanisms of ether-lipid action include effects on levels of intracellular protein phosphorylation, and disruption of cellular lipid metabolism. Normal cells typically possess the means to avoid or overcome the potentially toxic effects of ether-lipids, while cancer cells do not.

Thus far, synthetic ether lipids have been used for the treatment of different types of solid tumors such as brain tumors or mamma carcinomas (cf., for example, the German Patent DE 2619686 as well as the International Patent Applications WO 99/59599 and WO 00/01392, respectively). However, although the anti-tumor activity of these synthetic ether lipids has been experimentally proven in several animal tumor models, their clinical use is often hampered by systemic cytotoxic effects including hemolysis, particularly in the gastrointestinal tract but also inter alia in lung, liver or kidney.

Currently, in the vast majority of clinical trials on synthetic ether lipids the compounds are administered to patients orally or by using the intravenous route. In this context, it was found that the intravenous administration of a liposomal formulation and a lipophilic oil-in-water emulsion, respectively, is advantageous as compared to the free compound in order to improve therapeutic efficacy while reducing nonspecific toxicity in vivo (see, for example, Ahmad, I. et al. (1997) Cancer Res. 57, 1915-1921 as well as International Patent Application WO 91/09590).

However, it is also known in the art that certain ether phospholipid and carbamoyl salts while exhibiting benefits to a patient as competitive inhibitors of PAF or tumor growth with single or repeated injections, cause detrimental effects in the area of the injection. These detrimental effects are evident as lysis of red blood cells, severe edema, inflammation, and injection site-necrosis. These adverse effects are also called “detergent” effects. Where repeated injections are required, these detrimental effects are particularly disadvantageous as they render the sites of administration unsuitable and require fresh sites. Since the number of suitable sites on a patient is limited, it would be highly desirable to avoid said detrimental effects associated with intravenous administration of 1-O-octadecyl-2-O-methyl-glycero-3-phosphocholine.

Rather recently, it has been shown that in order to limit systemic side effects it is also possible to administer synthetic ether lipids orally together with a liquid drinkable vehicle. In the International Patent Application WO 99/59599, it is described that 1-O-octadecyl-2-O-methyl-glycero-3-phosphocholine can be administered together with water- or milk-based vehicles containing at least 3% (w/w) fat and/or protein. It is tempting to speculate that an efficient binding of 1-O-octadecyl-2-O-methyl-glycero-3-phosphocholine to the proteins and/or other lipids “mask” the ether-lipid thus resulting in a reduction of adverse side effects.

Nevertheless, in 10-20% of the patients treated with such water- and/or milk-based vehicles significant gastrointestinal incompatibilities (corresponding to WHO toxicity grades III and IV, respectively) have been observed that are associated with loss of appetite, nausea and/or vomiting, diarrhea, constipation or the like (see, for example, Drings, P. et al. (1992) Onkologie 15, 375-382).

Thus, there remains a need for a medicament for the treatment of hematological malignancies that overcomes the above limitations. In particular, there is a need for a medicament, which allows for an easy and convenient administration and provides the required pharmaceutical efficacy without significant side effects.

Accordingly, it is an object of the present invention to provide such a medicament for the treatment of hematological malignancies.

This object is achieved by the use of a tri-substituted glycerol compound having the features of independent claim 1 for the manufacture of a corresponding medicament that can be used individually or in combination with one or more other pharmaceuticals. Some of the preferred embodiments of the present invention are defined by the subject matter of the dependent claims.

According to the present invention, it has been found that tri-substituted glycerol compounds such as 1-O-octadecyl-2-O-methyl-glycero-3-phosphocholine are not only suitable for the use as a medicament for the selective treatment of hematological malignancies but also enhance the cytostatic and/or cytotoxic effects of several known chemotherapeutics on malignant cells in a synergistic manner. Thus, these synergistic effects allow for the administration of reduced drug doses in order to achieve the same pharmaceutical efficacy as compared to an individual administration, which will again reduce the risk of observing systemic adverse side effects.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%.

In a first aspect, the present invention relates to the use of a tri-substituted glycerol compound according to formula (I) or an enantiomer, diastereomer or pharmaceutically acceptable salt thereof for the manufacture of a medicament for the treatment of hematological malignancies,

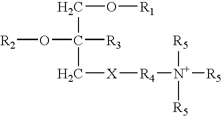

wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is selected from the group consisting of hydrogen and methyl.

The tri-substituted glycerol compound according to the invention may be used in amorphous or in crystalline form. The term “amorphous”, as used herein, refers to a solid in which there is no long-range order of the positions of the atoms, i.e. a non-crystalline material. In preferred embodiments of the invention, the tri-substituted glycerol compound is present in crystalline form.

The terms “Cₙ alkyl”, “Cₙ hydroxyalkyl”, and “Cₙ cycloalkyl”, as used herein, denote an alkyl group, a hydroxyalkyl group or a cycloalkyl group having n carbon atoms, respectively. For example, the term “C₁₈ alkyl” refers to an alkyl group having 18 carbon atoms. The alkyl groups or hydroxyalkyl groups according to the invention may be straight or branched.

The tri-substituted glycerol compounds of formula (I) have one or more asymmetric centers and thus they can exist as enantiomers or diastereomers. Accordingly, the medicament used in the present invention may comprise either one or more separate individual isomers (such as the L form and the D form) or mixtures of isomers, preferably racemic mixtures.

The tri-substituted glycerol compounds of formula (I) may be comprised in the medicament as pharmaceutically acceptable salts. Such salts may comprise any pharmaceutically acceptable anion “neutralizing” the positive charge of the nitrogen (e.g. chloride, bromide or iodide) or any pharmaceutically acceptable cation “neutralizing” the negative charge of the phosphate or sulfate moiety (e.g. sodium or potassium cations).

In a particular preferred embodiment of the present invention, the medicament comprises a tri-substituted glycerol compound according to formula (I), wherein X is phosphate, R₁ is —(CH₂)₁₇—CH₃, R₂ is CH₃, R₃ is H, R₄ is —(CH₂)₂—, and R₅ is CH₃.

The medicament according to the present invention may be any pharmaceutical dosage form that is therapeutically effective. Examples of such pharmaceutical dosage forms include inter alia tablets, pills, capsules, suspensions, emulsions, injection or infusion solutions, tinctures, powders and the like.

The medicaments used in the present invention comprise at least one pharmaceutically acceptable excipient. The term “pharmaceutically acceptable excipient”, as used herein, can be any substance used for the preparation of pharmaceutical dosage forms such as coating materials, film-forming materials, fillers, disintegrating agents, release-modifying materials, carrier materials, diluents, binding agents and other adjuvants, all of them well known in the art (cf. the references cited below). Preferably, the excipient used in the invention comprises at least one filler, at least one binder, at least one disintegrating agent, at least one flowability-controlling agent, and at least one lubricant.

The medicament may be administered via any parenteral or non-parenteral route. Parenteral application methods comprise, for example, intracutaneous, subcutaneous, intramuscular or intravenous injection and infusion techniques. Non-parenteral delivery modes include, for instance, oral or topical administration. Furthermore, the medicament may be administered locally or systemically.

Preferably, the medicament employed in the present invention is a solid dosage form, particularly preferably a pharmaceutical solid formulation suitable for oral application. Examples of such dosage forms include inter alia tablets, pills, capsules, granulates, pellets, powders, multi-particulate formulations (e.g., beads, granules or crystals), and dragees. The unit doses of multi-particulates may be incorporated into a pharmaceutical solid dosage formulation, e.g. via compression or shaping into tablets or by placing a requisite amount inside a gelatin capsule.

All these solid dosage forms for oral application as well as methods for their preparation are well established in the art (see, e.g., Gennaro, A. L. and Gennaro, A. R. (2000) Remington: The Science and Practice of Pharmacy, 20th Ed., Lippincott Williams & Wilkins, Philadelphia, Pa.; Ritschel, W. A. & Bauer-Brandl, A. (2002) Die Tablette Handbuch der Entwicklung, Herstellung und Qualitätssicherung. Editio-Cantor Verlag, Aulendorf, Germany; Crowder, T. M. et al. (2003) A Guide to Pharmaceutical Particulate Science. Interpharm/CRC, Boca Raton, Fla.; Stricker, H. (2003) Arzneiformenentwicklung, Springer Verlag, Berlin, Germany; Niazi, S. K. (2004) Handbook of Pharmaceutical Manufacturing Formulations, CRC Press, Boca Raton, Fla.).

In preferred embodiments of the invention, the pharmaceutical solid dosage form is selected from the group consisting of tablets, pills, capsules, and granules, with tablets being particularly preferred.

In another preferred embodiment of the invention, the solid dosage form is an enteric dosage form. That is, the dosage form remains stable in the stomach, i.e. in a highly acidic environment, with pH values in the range of <2.5. This may be achieved by providing a solid dosage form comprising a film coating. For example, the inventive dosage form may be in the form of a so-called film tablet.

Methods for the preparation of film coated dosage forms are also well established in the art (see, for example, Gennaro, A. L. and Gennaro, A. R. (2000) Remington: The Science and Practice of Pharmacy, 20th Ed., Lippincott Williams & Wilkins, Philadelphia, Pa.; Ritschel, W. A. & Bauer-Brandl, A. (2002) Die Tablette: Handbuch der Entwicklung, Herstellung and Qualitätssicherung. Editio-Cantor Verlag, Aulendorf, Germany; Crowder, T. M. et al. (2003) A Guide to Pharmaceutical Particulate Science. Interpharm/CRC, Boca Raton, Fla.; Niazi, S. K. (2004) Handbook of Pharmaceutical Manufacturing Formulations, CRC Press, Boca Raton, Fla.). Furthermore, the skilled artisan also knows how to provide film coatings with specific properties, like enteric coatings, film coating which dissolve upon contact with body fluids, controlled release coatings, taste-masking coatings or disintegrating coatings. In a particularly preferred embodiment, the solid dosage form of the invention comprises an enteric coating.

According to the present invention, it is to be understood that the tri-substituted glycerol compound is present in the medicament in any amount being effective to achieve the desired pharmacological effect such as to stop tumor progression or to induce an apoptotic effect in tumor cells when administered to a patient. Effective amounts are generally chosen in accordance with a number of factors, e.g., the age, size and general condition of the patient and the medical condition being treated, and determined by a variety of means, for example, dose ranging trials, well known to, and readily practiced by persons of ordinary skill in art given the teachings of this invention.

Typically, in the medicament according to the present invention the amount of the tri-substituted glycerol compound according to formula (I) is less than 400 mg, preferably it is in the range of 30 to 250 mg, and most preferably it is in the range of 50 to 150 mg. In particularly preferred embodiments of the invention, the amount of the tri-substituted glycerol compound according to formula (I) in the medicament is 75 mg and 100 mg, respectively.

The daily dosage of the tri-substituted glycerol compound in the medicament administered to a patient is less than 1200 mg, typically less than 900 mg, preferably in the range of 30 to 600 mg, more preferably in the range of 40 to 400 mg, and most preferably in the range of 50 to 350 mg. In specific embodiments, the daily dosage is 75, 100, 150, 200, 225, and 300 mg. Preferably, the daily dosage of the tri-substituted glycerol compound is administered as a single dose such as in form of one up to four tablets or capsules. However, it may also be possible to administer the compound in multiple doses such as two or three individual doses administered during the day, e.g. in the morning, at noon, and at night.

The term “hematological malignancies” (also referred to as “hematological neoplasms”), as used herein, denotes any type of cancer that affects blood, bone marrow or lymphatic organs. Hematological malignancies include inter alia leukemia, lymphoma, multiple myeloma, and myelodysplastic syndrome.

The term “leukemia”, as used herein, refers to any cancer of the blood or bone marrow characterized by an abnormal proliferation of blood cells, usually of white blood cells (i.e. leukocytes). Within the scope of the present invention, the term includes any acute and chronic forms of leukemia as well as any form of lymphoid or myeloid leukemia. Forms of lymphoid (or lymphocytic) leukemia are characterized in that lymphoid cells (that is, agranulocytes) such as lymphocytes and monocytes are affected, whereas when myeloid cells (that is, granulocytes) such as eosinophils, neutrophils, and basophils are affected, the disease is referred to as myeloid (or myelogenous) leukemia. In preferred embodiments of the invention, the leukemia is selected from the group consisting of acute myeloid leukemia (AML), acute lymphoid leukemia (ALL), chronic myeloid leukemia (CML), and chronic lymphoid leukemia (CLL).

The term “lymphoma”, as used herein, refers to any form of cancer originating from lymphocytes, that is it includes Hodgkin lymphoma characterized by the orderly spread of the disease from one lymph node and the presence of Reed-Sternberg cells as well as to any type of non-Hodgkin lymphoma. In preferred embodiments of the invention, the hematological malignancy is selected from the group consisting of Hodgkin lymphoma and non-Hodgkin lymphoma.

The term “multiple myeloma” (also referred to as “myeloma” or “plasmacytoma”), as used herein, denotes a type of cancer of plasma cells, i.e. the immune cells in bone marrow that produce antibodies.

The term “myelodysplastic syndrome” (formerly known as “pre-leukemia”), as used herein, denotes a diverse collection of hematological conditions united by ineffective production of blood cells and varying risks of transformation to acute myelogenous leukemia. Anemia requiring chronic blood transfusion is frequently present. Although not a true malignant neoplasm, myelodysplastic syndrome is nevertheless classified within the hematological neoplasms.

The medicament according to the invention may be used for the treatment of hematological malignancies individually or in combination with at least one other pharmaceutical comprising at least one additional active ingredient. That is, an adjunction is also within the scope of the present invention, wherein a medicament comprising a tri-substituted glycerol compound is used together with at least one other pharmaceutical comprising one or more additional active ingredients, i.e. active ingredients different from the tri-substituted glycerol compounds as defined in claim 1. In preferred embodiments of the invention, the one or more additional active ingredients are selected from the group consisting of anti-neoplastic agents and/or from the group consisting of antibodies directed against one or more epitopes on the cell surface of hematological (hematopoetic) cells.

The term “anti-neoplastic agents” (herein also referred to as “cytostatic drugs”), as used herein, refers to any pharmaceuticals which interfere with cellular karyokinesis (i.e. mitosis) and/or cytokinesis (i.e. cell division), in particular in malignant cells (i.e. tumor cells), that is which cause an interruption, a delay or a prohibition of said process(es). Also included herein are cytostatic drugs having a cytotoxic effect as well, that is pharmaceuticals causing cell death by interfering with karyokinesis and/or cytokinesis.

Examples of anti-neoplastic agents (cytostatic drugs) that can be used in the present invention include inter alia alkylating agents, antimetabolites, plant alkaloids, antibiotics, inhibitors of topoisomerases I and/or II, steroids, inhibitors of tyrosine kinases, proteosome inhibitors, and DNA intercalating agents.

The term “alkylating agents”, as used herein, denotes compounds being capable of adding alkyl groups to various electronegative groups present in cellular molecules such as nucleic acids or proteins. Typically, these compounds interfere with tumor growth and progression by cross-linking guanine nucleobases in DNA strands. This modification prevents DNA uncoiling and strand separation, and thus DNA replication.

In preferred embodiments of the invention, the alkylating agents are selected from the group of nitrogen mustards, nitrosoureas, platinum derivatives, and alkylsulfonates. Examples of nitrogen mustards include inter alia chlorambucil (CLL), cyclophosphamide (lymphomas), melphalan (multiple myeloma), and mechlorethamine (Hodgkin lymphomas). An example of nitrosoureas is inter alia carmustine (lymphomas), and examples of platinum derivatives include inter alia cisplatin (lymphomas) and carboplatin (lymphomas, ALL). Examples of alkylsulfonates include inter alia busulfan (CML), and treosulfan (lymphomas).

The term “antimetabolite”, as used herein, refers to chemical compounds having a similar structure as a substance (a metabolite) that is required for cellular biochemical reactions, yet different enough to interfere with the normal cellular function.

In preferred embodiments of the invention, the antimetabolites are selected from the group consisting of methotrexate, (ALL), purine analogs, and pyrimidine analogs. Examples of purine analogs include inter alia fludarabine (non-Hodgkin lymphomas, AML), clofarabine (ALL), pentostatine (CLL), and cladribine (CLL), and examples of pyrimidine analogs include inter alia cytarabine (leukemia, non-Hodgkin lymphomas), and decitabine (myelodysplastic syndrome).

Examples of plant alkaloids (i.e. naturally occurring or synthetically produced plant amines) that can be used in the present invention include inter alia vincristine (non-Hodgkin lymphoma, ALL) and vinblastine (Hodgkin lymphomas). Exemplary antibiotics that can be used in the invention include inter alia mitoxantrone (AML), epirubicin (lymphomas), doxorubicin (Hodgkin lymphoma), daunorubicin (AML, ALL), and bleomycin (Hodgkin lymphoma). Topoisomerase inhibitors interfere with normal function of topoisomerases type I and/or type II, that is enzymes that act on DNA topology. Within the scope of the present invention, topoisomerase II inhibitors such as etoposide (lymphomas, AML, CML) or teniposide (ALL) are preferred.

Further anti-neoplastic agents to be used in the invention include inter alia the steroid dexamethasone (non-Hodgkin lymphomas), the tyrosine kinase inhibitor imatinib (CML), the proteasome inhibitor bortezomib (multiple myeloma), and the DNA intercalating agents amsacrine (ALL, AML), and thalidomide (multiple myeloma).

The respective primary medical use of the above anti-neoplastic agents within the scope of the present invention is indicated in brackets. However, this does not preclude the use of any of these drugs for the treatment of any other hematological malignancies. Furthermore, it is also possible to use a combination of two or more of these drugs for the treatment of any hematological malignancy.

In a particular preferred embodiment of the present invention, the anti-neoplastic agents are selected from the group consisting of chlorambucil, cyclophosphamide, melphalan, mechlorethamine, carmustine, cisplatin, carboplatin, busulfan, treosulfan, methotrexate, fludarabine, clofarabine, pentostatine, cladribine, cytarabine, decitabine, vincristine, vinblastine, mitoxantrone, epirubicin, doxorubicin, daunorubicin, bleomycin, etoposide, teniposide, dexamethasone, imatinib, bortezomib, amsacrine, and thalidomide.

The antibodies used in the present invention comprise monoclonal and polyclonal antibodies directed against one or more epitopes on the cell surface of hematological (hematopoetic) cells. The term “hematological (hematopoetic) cells”, as used herein, denotes any cells of hematopoetic origin including cells of myeloid and lymphatic origin. The term “directed against one or more epitopes on the cell surface”, as used herein, refers to the fact that an antibody used in the present invention may recognize/bind one or more cell surface molecules (i.e. epitopes) of a hematological cell.

In preferred embodiments of the invention, the antibody is a monoclonal antibody, that is an antibody directed against a specific antigen that is generated from a population of B-cell lymphocytes that are all duplicates descended from one original antibody producing cell. The antibody may be a human antibody or a chimeric (humanized) antibody, for example a chimeric murine/human antibody. Methods for the generation of such “engineered” monoclonal antibodies are well established in the art (reviewed, e.g., in Coligan, J. E. et al. (2002) Current Protocols in Immunology, Wiley & Sons, Hoboken, N.J.)

Examples of such monoclonal antibodies that can be employed in the present invention include inter alia alemtuzumab (anti-CD52; CLL, lymphomas), rituximab (anti-CD20; CLL, non-Hodgkin-lymphoma), gentuzumab ozogamizin (anti-CD33; AML)

In a second aspect, the present invention relates to a corresponding method for the prevention and/or the treatment of hematological malignancies, wherein the method comprises:
(a) administering to a patient a medicament or a combination of medicaments as defined in the invention.

As outlined above, the medicament or combination of medicaments according to the present invention may be administered via any parenteral or non-parenteral route. In case a combination of medicaments is used, the individual pharmaceuticals may be administered via the same or via different routes. Preferably, the medicament comprising the tri-substituted glycerol compound is administered orally, particularly preferably as a solid dosage form. Furthermore, the medicament is preferably administered as a single dose such as in form of one tablet or capsule per day. However, it may also be possible to administer the medicament in multiple doses such as two or three individual doses administered during the day. In case a combination of medicaments is used (such as in form of a kit-of-parts), the individual pharmaceuticals (i.e. the medicament comprising the tri-substituted glycerol compound and the at least one other pharmaceutical) may be administered simultaneously, separately or sequentially. For example, it may be possible to administer one medicament during a meal, and another one or more hours later.

In a preferred embodiment of the invention, the method further comprises:
(b) determining the extent of Fas receptor gene expression in one or more malignant cells of the patient to be treated before administering the medicament or the combination of medicaments.

As already outlined above, the cell death receptor Fas (also known as APO-1 or CD95) has been identified as a cellular target of 1-O-octadecyl-2-O-methyl-glycero-3-phosphocholine (cf., for example, Gajate, C. et al. (2004) J. Exp. Med. 200, 353-365; Nieto-Miguel, T. et al. (2006) J. Biol. Chem. 281, 14833-14840). In the context of the present invention, it has been found that malignant hematological cells (i.e. tumor cells) over-expressing the Fas receptor display an increased sensitivity towards the treatment using a medicament as defined in the claims.

The terms “expression” or “gene expression”, as used herein, relate to the entirety of regulatory pathways converting the information encoded in the nucleic acid sequence of a gene first into messenger RNA (mRNA) and then to a protein. Accordingly, the expression of a gene comprises its transcription into a primary hnRNA, the processing of this hnRNA into a mature RNA and the translation of the mRNA sequence into the corresponding amino acid sequence of the protein.

As used herein, the term “over-expressing” refers to an expression level of at least 100% of that of a non-malignant (non-tumorgenic) control cell (i.e. a wild-type cell). Accordingly, in one embodiment of the invention, the method further comprises comparing results of the measurements obtained in the tumor cells with those obtained in one or more control cells of a healthy subject.

The term “determining” refers to any method to detect a “gene expression product”. Within the scope of the present invention, the term “gene expression product” refers not only to the protein encoded by that gene but also to the respective mRNA, which may be regarded as the “first gene product” during the course of gene expression. These methods comprise established standard procedures well known in the art (cf., for example, Sambrook, J. et al. (1989) Molecular Cloning: A Laboratory Manual. 2nd ed., Cold Spring Harbor Laboratory Press, Cold Spring Harbor, N.Y.; Ausubel, F. M. et al. (2001) Current Protocols in Molecular Biology. Wiley & Sons, Hoboken, N.J.). Examples of such methods are RT-PCR, RNAse protection assay, Northern analysis, Western analysis, ELISA, radioimmunoassay or fluorescence titration assay.

For example, an mRNA can be detected by hybridization with a labeled nucleic acid probe (DNA or RNA) and subsequent analysis using either Northern blotting or performing an RNAse protection assay. Alternatively, the respective cDNA can be prepared from cellular RNA (either total RNA or mRNA) by reverse transcription and analyzed for the absence or presence of a particular nucleic acid species by PCR amplification (i.e. RT-PCR). Proteins can be conveniently detected by using specific antibodies, which may be monoclonal or polyclonal antibodies. For visualization, the antibody may be labeled or a secondary antibody conjugated to an appropriate label may be used, which binds to the specific primary antibody. Detection may be done, for example, in a Western blot-analysis or an ELISA. Suitable labels for performing the methods of the invention include enzyme labels, radioactive labels, fluorescent labels, chromogenic labels, luminescent labels, digoxigenin, biotin, small organic molecules, metals, metal complexes, and colloidal gold.

In another preferred embodiment, the method further comprises:
(c) determining one or more of the following in the one or more malignant cells of the patient to be treated before administering the medicament or the combination of medicaments:
   (i) the presence or absence of one or more ras gene mutations;
   (ii) the presence or absence of one or more FGFR3 gene mutations; and
   (iii) the presence or absence of one or more PTEN gene mutations.

In one embodiment, the method further comprises comparing results of the measurements obtained in the one or more malignant cells with those obtained in one or more control cells, i.e. non-tumorgenic cells, of a healthy subject.

The term “ras gene mutation”, as used herein, refers to any genetic variation of a member of the ras gene family, i.e. the H-ras, K-ras, and N-ras genes, in particular of the human ras genes (see Land, H. et al. (1983) Science 222, 771-778). Genetic variations according to the present invention include the addition, the deletion, and the substitution of one or more nucleotides of the respective DNA sequence. Preferably, such one or more mutations give rise to an altered amino acid sequence of the respective corresponding Ras protein, which also includes the formation of truncated or extended protein variants. Such mutations may also affect protein function. For example, they may cause a (constitutive) activation of the Ras protein or may result in a dominant-negative Ras variant. Examples of such ras gene mutations include inter alia mutations affecting codons 12, 13, and 61 of human N-ras as well as codons 12 and 71 of human K-ras (cf. the references cited below).

In preferred embodiments of the invention, the one or more ras gene mutations cause an activation of the corresponding Ras protein (i.e. an increase of Ras protein activity). Particular preferred examples of such ras gene mutations comprise inter alia mutations at codon 61 of human N-ras including CAA→CGA and CAA→CAC as well as mutations at codon 12 of human K-ras including GGT→GCT and GGT→GAT (cf., e.g., Bos, J. L. et al. (1984) Nucl. Acids Res. 12, 9155-9163; Pilz, R. B. et al. (1997) Cell Growth Diff. 8, 53-59; Delgado, M. D. et al. (2000) Oncogene 19, 783-790; Chesi, M. et al. (2001) Blood 97, 729-736).

The term “FGFR3 gene mutation”, as used herein, refers to any genetic variation of the fibroblast growth factor receptor 3 (FGFR3) gene, in particular of the human FGFR3 gene (Ornitz, D. M., and Itoh, N. (2001) Genome Biol. 2, reviews 3005.1-3005.12). Genetic variations according to the present invention include the addition, the deletion, and the substitution of one or more nucleotides of the respective DNA sequence. Preferably, such one or more mutations give rise to an altered amino acid sequence of the respective corresponding FGFR3 protein, which also includes the formation of truncated or extended protein variants. Such mutations may also affect protein function. For example, they may cause a (constitutive) activation of the FGFR3 protein or may result in a dominant-negative FGFR3 variant. Examples of such FGFR3 gene mutations include inter alia mutations affecting codons 373, 384, and 650 of the human FGFR3 as well as a deletion of the human FGFR3 stop codon (cf. the references cited below).

In preferred embodiments of the invention, the one or more FGFR3 gene mutations cause an activation of the corresponding FGFR3 protein (i.e. an increase of FGFR3 protein activity). Particular preferred examples of such FGFR3 gene mutations comprise inter alia mutations of human FGFR3 at codon 373 including TAT→TGT, at codon 650 including AAG→GAG, and a deletion of the human FGFR3 stop codon (cf., e.g., Chesi, M. et al. (1997) Nat. Genet. 16, 260-264; Chesi, M. et al. (2001) Blood 97, 729-736).

In particular preferred embodiments, the presence or absence of the one or more FGFR3 gene mutations are determined in the concomitant presence of the t(4;14) chromosomal translocation. That is, the sequence of the FGFR3 gene is analyzed in one or more malignant cells (such as hematopoetic cells) bearing said chromosomal translocation.

The term “t(4;14) chromosomal translocation”, as used herein, denotes a karyotypically silent genetic translocation, i.e. an abnormal chromosomal rearrangement caused by the interchange of parts between non-homologous chromosomes, on human chromosome 14, in particular it denotes the t(4;14)(p16;q32) translocation, which results in a dysregulated expression of the fibroblast growth factor receptor 3 gene (FGFR3 gene) (see, for example, Richelda, R. et al. (1997) Blood 90, 4062-4070; Chesi, M. et al. (2001) Blood 97, 729-736).

The presence of the t(4;14) chromosomal translocation in the one or more malignant cells to be analyzed may inter alia be determined by classical Southern analyses (cf., for example, Sambrook, J. et al. (1989), supra; Ausubel, F. M. et al. (2001), supra) or by in situ hybridization techniques, preferably be fluorescence in situ hybridization (FISH) (for review see, for example, van der Ploeg, M. (2000) Eur. J. Histochem. 44, 7-42; Levsky, J. M. and Singer, R. H. (2003) J. Cell Sci. 116, 2833-2838).

The term “PTEN gene mutation”, as used herein, refers to any genetic variation of the PTEN tumor suppressor gene, in particular of the human PTEN gene (see, e.g., Sakai, A. et al. (1998) Blood 92, 3410-3415; Wu, H. et al. (2003) Oncogene 22, 3113-3122). Genetic variations according to the present invention include the addition, the deletion, and the substitution of one or more nucleotides of the respective DNA sequence. Preferably, such one or more mutations give rise to an altered amino acid sequence of the respective corresponding PTEN protein, which also includes the formation of truncated or extended protein variants. Such mutations may also affect protein function. For example, they may cause a (constitutive) activation of the PTEN protein or may result in a dominant-negative PTEN variant.

Preferred examples of PTEN gene mutations to be determined in the present invention include inter alia a deletion of exons 2-5 of the human PTEN gene, a 2 by (base pairs) deletion and 9 by insertion at codon 234, and a 39 by insertion at codon 246 of the human PTEN gene sequence, respectively.

The respective mutations of the ras, FGFR3, and PTEN genes are determined by established standard laboratory methods, typically by polymerase chain reaction (PCR), restriction analyses and/or DNA sequence analyses (cf., for example, Sambrook, J. et al. (1989), supra; Ausubel, F. M. et al. (2001), supra).

Instead of or in addition to determining the absence or presence of one or more mutations of the ras, FGFR3, and/or PTEN genes, respectively, which all affect MAPK/ERK- and/or PI3K/AKT-signaling (reviewed, e.g., in Johnson, G. L. and Lapadat, R.(2002) Science 298, 1911-1913; Lee, J. T., and McCubrey, J. A. (2002) Leukemia 16, 486-507; Platanias, L. C. (2003) Blood 101, 4667-4679; Meier, F. et al. (2005) Front. Biosci. 10, 2986-3001; Martelli, A. M. et al. (2006) Leukemia 20, 911-928) it may also be possible within the scope of the present invention to determine the “activity status” (i.e. activation or not) of the respective signaling pathway “as such”. For example, an activation of the MAPK/ERK pathway may be determined by measuring the phosphorylation of ERK-1/2 by immunochemical methods well known in the art such as Western analysis, immune precipitation, and ELISA, preferably by employing phosphorylation-specific anti-ERK-1/2 antibodies commercially available by various suppliers (for a review of immunological methods see, e.g., Coico, R. et al. (2006) Current Protocols in Immunology. Wiley & Sons, Hoboken, N.J.).

In a third aspect, the present invention relates to an in vitro method for determining the susceptibility of one or more malignant cells to a medicament or a combination of medicaments as defined in the invention, the method comprising:
(a) determining the extent of Fas receptor gene expression in the one or more malignant cells.

In a preferred embodiment of the invention, the method further comprises:
(b) determining one or more of the following in the one or more malignant cells:
   (i) the presence or absence of one or more ras gene mutations;
   (ii) the presence or absence of one or more FGFR3 gene mutations; and
   (iii) the presence or absence of PTEN gene mutations.

In preferred embodiments of the invention, the presence or absence of the one or more FGFR3 gene mutations is determined in the concomitant presence of the t(4;14) chromosomal translocation.

Optionally, the method further comprises:
(c) comparing the results of the measurements obtained in (a) and/or (b) with those obtained in one or more control cells.

In a forth aspect, the present invention relates to a tri-substituted glycerol compound as defined herein for the treatment of hematological malignancies. In preferred embodiments, the hematological malignancies are selected from the group consisting of leukemia, lymphoma, multiple myeloma, and myelodysplastic syndrome.

In a fifth aspect, the present invention relates to a kit-of-parts comprising a medicament for the treatment of hematological malignancies, the medicament comprising a tri-substituted glycerol compound as defined in the invention.

In a preferred embodiment, the kit-of-parts further comprises another medicament comprising one or more additional active ingredients. Particularly preferably, the one or more additional active ingredients are either selected from the group of anti-neoplastic agents or are selected from the group of antibodies, wherein said antibodies are directed against one or more epitopes on the cell surface of hematological cells, as defined above.

The components (parts) of the kit (i.e. the individual medicaments or active ingredients) may be administered during a combination treatment (regimen) either simultaneously, separately or sequentially (cf., for example, Sambrook, J. et al. (1989), supra; Ausubel, F. M. et al. (2001), supra).

The invention is further described by the following figures and examples, which are solely for the purpose of illustrating specific embodiments of this invention, and are not to be construed as limiting the scope of the invention in any way.

Materials used in tests below are either commercially available or easily prepared from commercially available materials by those skilled in the art.

### FIGURES

FIG. 1 depicts the cytotoxic effects of ET-18-OCH3 (1-O-octadecyl-2-O-methyl-glycero-3-phosphocholine) on various tumor cell lines. The following cell lines were used: KMS-12-BM, K562, RPMI8226, OPM-2, NCI-H929, U266, and HL60. The cells were grown in 96-well tissue culture plates in a volume of 200 μl to a concentration of 1×10⁶ cells/ml. Then, the cells were incubated for 20 h with 5 μg/ml, 10 μg/ml, and 20 μg/ml ET-18-OCH3 (final concentrations), respectively. Cell death was assessed by staining the cells with 7-amino-actinomycin D (final concentration 200 μg/ml) and analyzed via flow cytometry as described (see, e.g., Schmid, I. et al. (1994) J. Immunol. Meth. 170, 145-157; Ayuk, F. A. et al. (2005) Exp. Hematol. 33, 1531-1536).

FIG. 2 depicts the cytotoxic effects of ET-18-OCH3 alone and in combination with bortezomib (Velcade®), an inhibitor of the 26S proteasome, on Jurkat cells. Cell culture was performed as described in FIG. 1. ET-18-OCH3 was added in a final concentration of 2.5 or 5.0 μg/ml (“E2.5” and “E5”, respectively) without or in combination with 5 or 10 ng/ml bortezomib (“Vel5” and “Vel10”, respectively), and the cells were incubated for 20 h. Cell death was assessed as described in FIG. 1. The results are expressed as mean±SEM of three independent experiments.

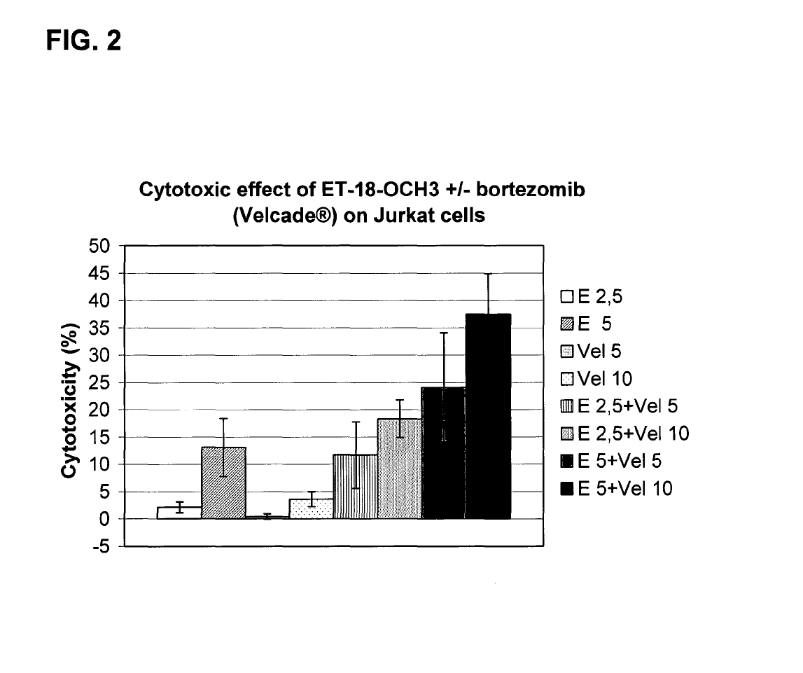

FIG. 3 depicts the cytotoxic effects of ET-18-OCH3 alone and in combination with bortezomib on HL60 cells. Cell culture, compound concentrations, and assay conditions were as described in FIG. 2. The results are expressed as mean±SEM of four independent experiments.

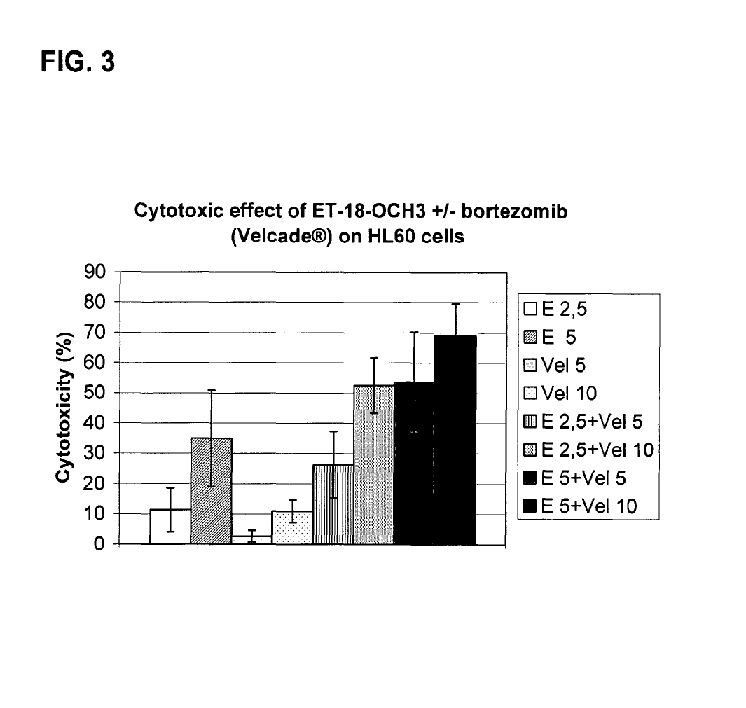

FIG. 4 depicts the cytotoxic effects of ET-18-OCH3 alone and in combination with bortezomib on RPMI8226. Cell culture, compound concentrations, and assay conditions were as described in FIG. 2. The results are expressed as mean±SEM of three independent experiments.

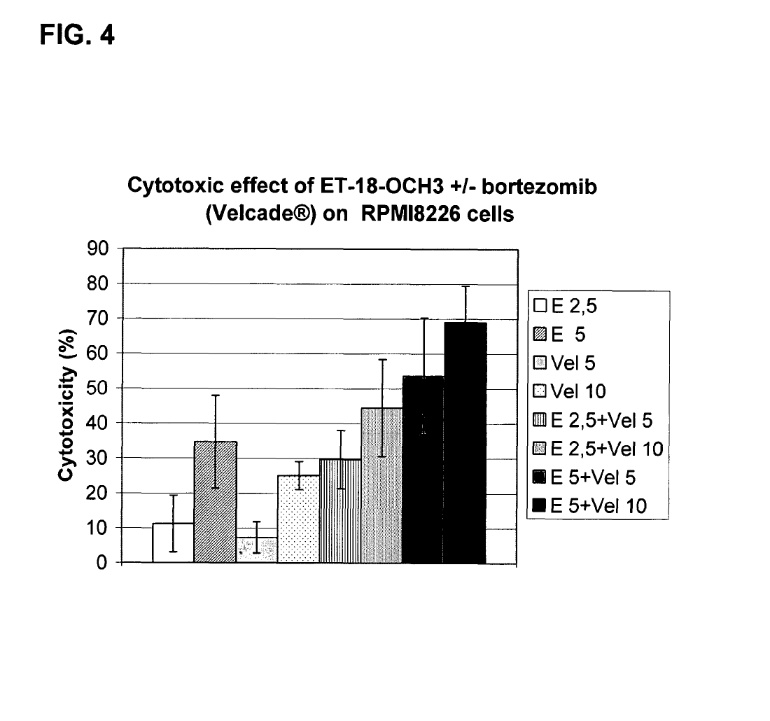

FIG. 5 depicts the cytotoxic effects of ET-18-OCH3 alone and in combination with fludarabine, a purine analog, on Jurkat cells. Cell culture was performed as described in FIG. 1. ET-18-OCH3 was added in a final concentration of 2.5 or 5.0 μg/ml (“E2.5” and “E5”, respectively) without or in combination with 10 or 50 μM fludarabine (“Flu10” and “Flu50”, respectively), and the cells were incubated for 20 h. Cell death was assessed as described in FIG. 1. The results are expressed as mean±SEM of three independent experiments.

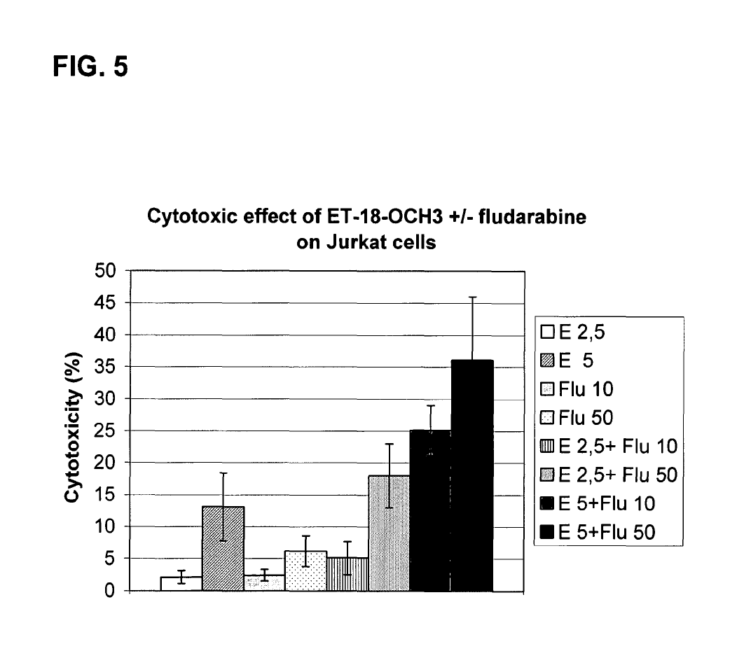

FIG. 6 depicts the cytotoxic effects of ET-18-OCH3 alone and in combination with fludarabine on HL60 cells. Cell culture, compound concentrations, and assay conditions were as described in FIG. 5. The results are expressed as mean±SEM of four independent experiments.

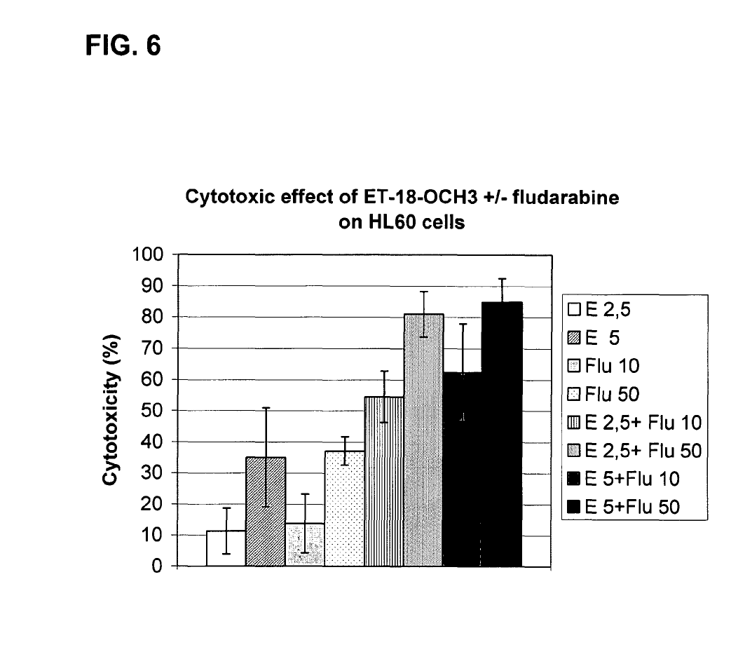

FIG. 7 depicts the cytotoxic effects of ET-18-OCH3 alone and in combination with cytarabine, a pyrimidine analog, on Jurkat cells. Cell culture was performed as described in FIG. 1. ET-18-OCH3 was added in a final concentration of 2.5 or 5.0 μg/ml (“E2.5” and “E5”, respectively) without or in combination with 10 or 40 μM cytarabine (“Ara-C 10” and “Ara-C40”, respectively), and the cells were incubated for 20 h. Cell death was assessed as described in FIG. 1. The results are expressed as mean±SEM of three independent experiments.

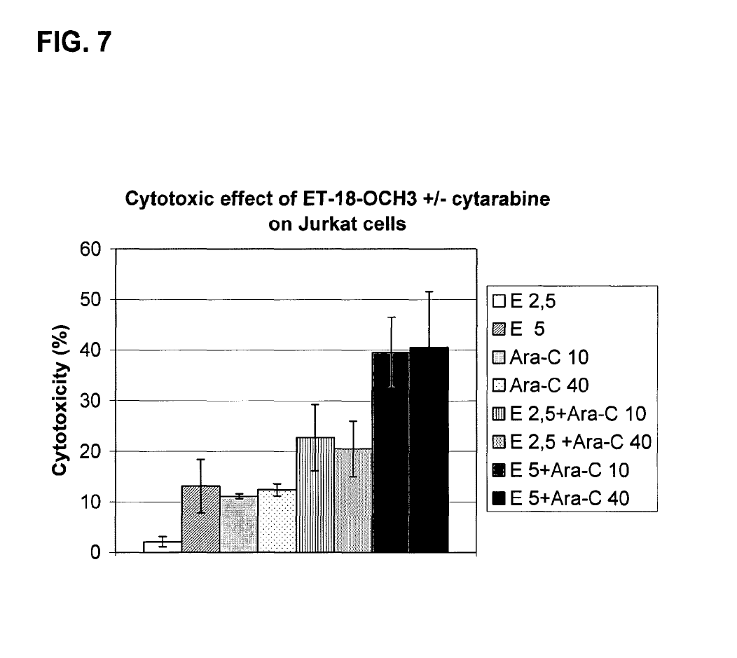

FIG. 8 depicts the cytotoxic effects of ET-18-OCH3 alone and in combination with cytarabine on HL60 cells. Cell culture, compound concentrations, and assay conditions were as described in FIG. 7. The results are expressed as mean±SEM of four independent experiments.

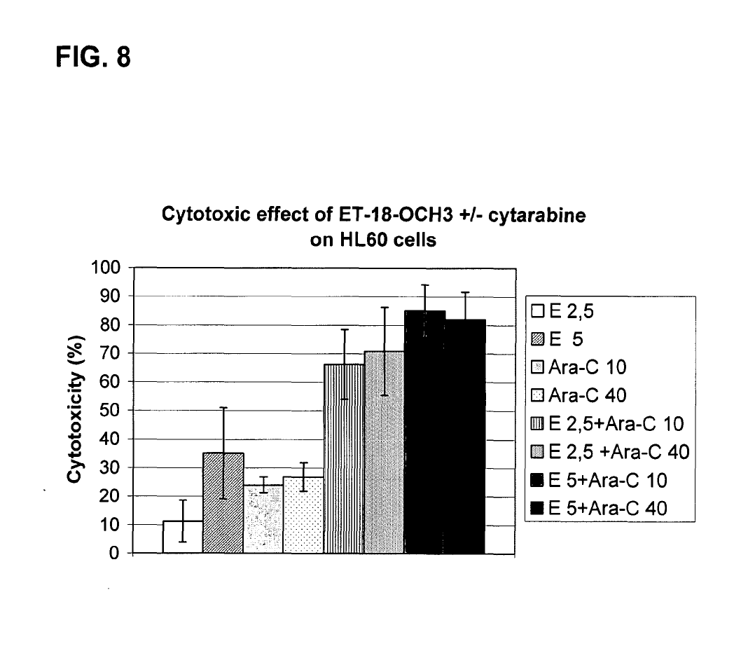

FIG. 9 depicts the cytotoxic effects of ET-18-OCH3 alone and in combination with etoposide, a topoisomerase II inhibitor, on Jurkat cells. Cell culture was performed as described in FIG. 1. ET-18-OCH3 was added in a final concentration of 2.5 or 5.0 μg/ml (“E2.5” and “E5”, respectively) without or in combination with 1.0, 2.5 or 5.0 μM etoposide (“Eto1”, “Eto2.5”, and “Eto5”, respectively), and the cells were incubated for 20 h. Cell death was assessed as described in FIG. 1. The results are expressed as mean±SEM of three independent experiments.

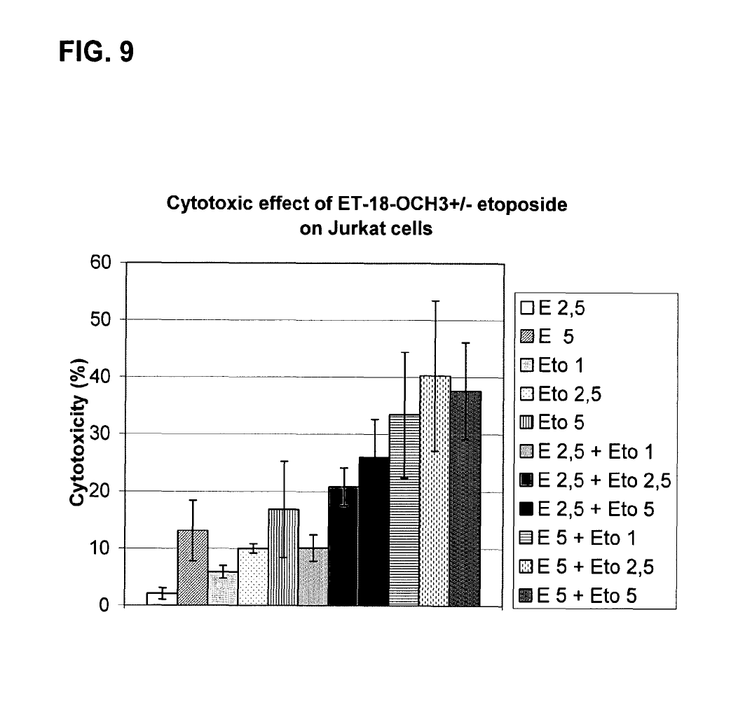

FIG. 10 depicts the cytotoxic effects of ET-18-OCH3 alone and in combination with etoposide on HL60 cells. Cell culture, compound concentrations, and assay conditions were as described in FIG. 9. The results are expressed as mean±SEM of four independent experiments.

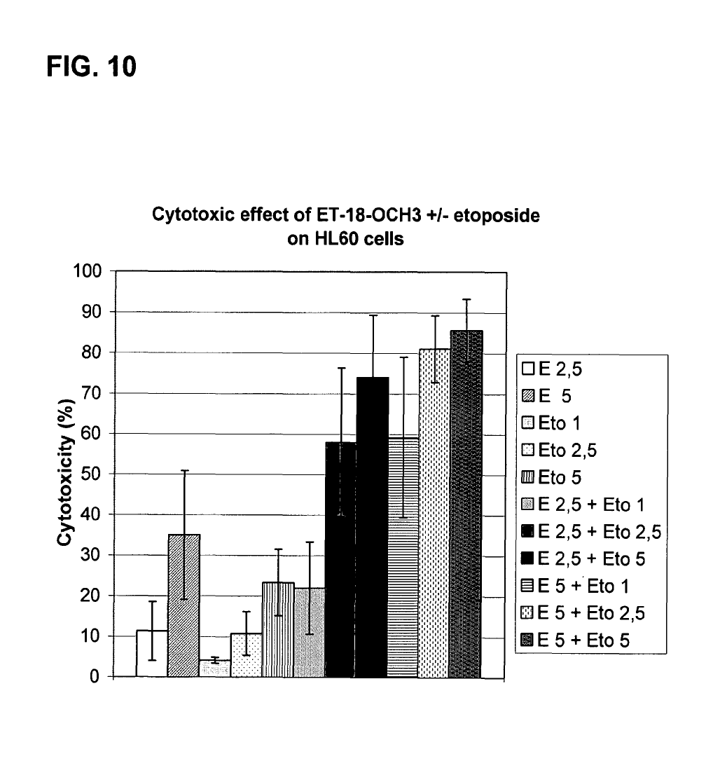

### EXAMPLES

### Example 1

### Determining the cytotoxic effect of 1-O-octadecyl-2-O-methyl-glycero-3-phosphocholine on various human hematological tumor cell lines

The following human cell lines were used:
1. KMS-12-BM (DMSZ no. ACC 551), bone marrow cells; myeloma/plasmacytoma; none of the genetic alterations defined herein; Chesi, M. et al. (2001), supra.
2. K562 (ATCC no. CCL-243); bone marrow cells; CML; none of the genetic alterations defined herein; Delgado, M. D. et al. (2001), supra.
3. RPMI8226 (ATCC no. CLL-155); peripheral blood cells; myeloma/plasmacytoma; K12-G>A K-ras gene mutation (GGT→GCT at codon 12); Chesi, M. et al. (2001), supra.
4. OPM-2 (ATCC no. CRL-13007); lymphoblast cells; myeloma; t(4;14) chromosomal translocation; K650E FGFR3 gene mutation (AAG→GAG at codon 650); Chesi, M. et al. (2001), supra.
5. NCI-H929 (ATCC no. CRL-9068); bone marrow cells; myeloma/plasmacytoma; t(4;14) chromosomal translocation; N13-G>D N-ras gene mutation (GGT→GAT at codon 13); Chesi, M. et al. (2001), supra.
6. U266 (ATCC no. TIB-196); peripheral blood cells; myeloma/plasmacytoma; none of the genetic alterations defined herein; Chesi, M. et al. (2001), supra.
7. HL60 (ATCC no. CCL-240); peripheral blood cells; AML; N61-Q>R N-ras gene mutation (CAA→CGA at codon 61); Bos, J. L. et al. (1984), supra.
8. Jurkat (ATCC no. TIB-152); peripheral blood cells; ALL; 2 by deletion and 9 by insertion at codon 234 in exon 7 of the PTEN gene (resulting in downstream stop codons); Sakai, A. et al. (1998), supra.

The cells were grown in 96-well tissue culture plates in a volume of 200 μl to a concentration of 1×10⁶ cells/ml. Then, the cells were incubated for 20 h with a final concentration of 5 μg/ml, 10 μg/ml, and 20 μg/ml 1-O-octadecyl-2-O-methyl-glycero-3-phosphocholine (ET-18-OCH3), respectively. Cell death was assessed by staining the cells with 7-amino-actinomycin D (Calbiochem, Darmstadt, Germany; final concentration 200 μg/ml) and analyzed via flow cytometry using a FACS-Scan flow cytometer (BD Biosciences, San Jose, Calif., USA) as described (see, e.g., Schmid, I. et al. (1994) J. Immunol. Meth. 170, 145-157; Ayuk, F. A. et al. (2005) Exp. Hematol. 33, 1531-1536).

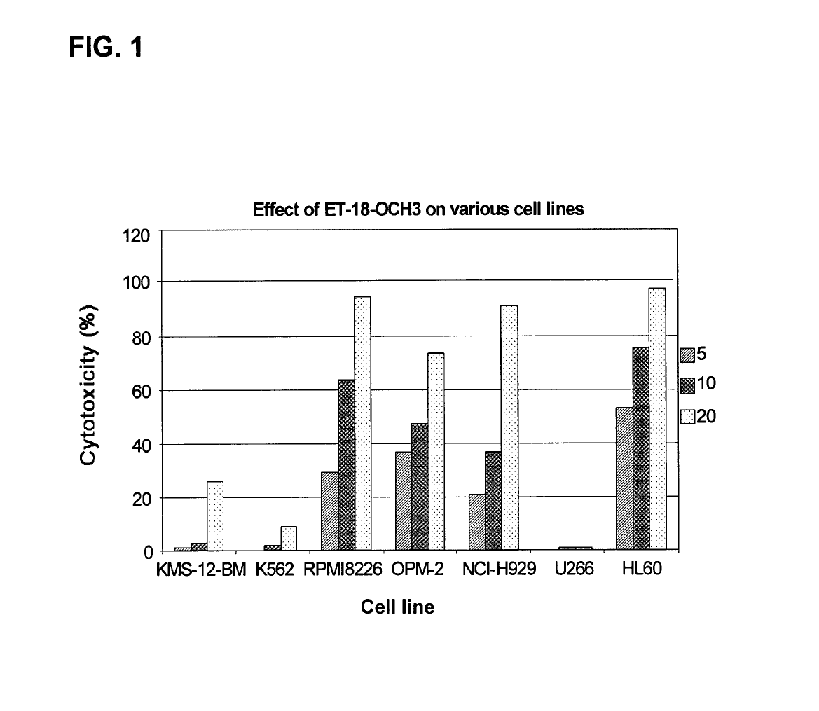

The results obtained in a typical experiment are summarized in FIG. 1. With the exception of U266 cells, which apparently do not respond to ET-18-OCH3 at all, when exposed to ET-18-OCH3 all cell lines showed a dose-dependent effect. The application of 20 μg/ml ET-18-OCH3 resulted in almost complete cell death in RPMI8226, NCI-H929, and HL60 cells, respectively. In OPM-2 cells about 75% of the cells died upon exposure to 20 μg/ml ET-18-OCH3. Interestingly, in those cell lines harboring none of the genetic alterations defined herein, namely the t(4;14) chromosomal translocation, ras, FGFR3, and PTEN gene mutations, respectively (i.e. in KMS-12-BM, K562, and U266), the cytotoxicity is significantly reduced or even almost abolished. These results demonstrate that the presence of one or more of the above genetic alterations is a strong indication for the susceptibility of a given cell to the treatment with ET-18-OCH3.

### Example 2

### Determining the cytotoxic effect of 1-O-octadecyl-2-O-methyl-glycero-3-phosphocholine in combination with different anti-neoplastic agents on various cell lines

The following cytostatic drugs were used: bortezomib (Velcade®; Janssen Cilag, Neuss, Germany), an inhibitor of the 26S proteasome that is commonly used for the treatment of multiple myeloma; fludarabine (Sigma, St. Louis, USA), a purine analog mainly used in the treatment of AML and CLL; cytarabine (also referred to as “cytosine arabinoside” or “Ara-C”; Sigma, St. Louis, USA), a pyrimidine analog mainly used in the treatment of AML and non-Hodgkin lymphomas; and etoposide (Sigma, St. Louis, USA), an inhibitor of topoisomerase II that is commonly used in the treatment of lymphomas.

The cells were again grown in 96-well tissue culture plates in a volume of 200 μl to a concentration of 1×10⁶ cells/ml. Then, the cells were incubated for 20 h with the respective drugs. The following final concentrations were used (in general, the concentrations employed resulted in less than 50% cytotoxicity when administered alone): 2.5 μg/ml and 5.0 μg/ml 1-O-octadecyl-2-O-methyl-glycero-3-phosphocholine (ET-18-OCH3); 5 ng/ml and 10 ng/ml bortezomib; 10 μM and 50 μM fludarabine; 10 μM and 40 μM cytarabine; and 1 μM, 2.5 μM, and 10 μM etoposide, respectively. The cytotoxic effect of ET-18-OCH3 in combination with bortezomib was determined in Jurkat, HL60 cells, and RPMI8226 cell, whereas the cytotoxic effect of ET-18-OCH3 in combination with cytarabine, fludarabine and etoposide, respectively, in Jurkat and HL60 cells. Cell death was assessed as described in example 1.

The results obtained are summarized in the following Tables 1 to 4 (the data represent means±SEM of 3 (Jurkat cells and RPMI8226 cells) or 4 independent experiments (HL60 cells):

TABLE 1
Effect of ET-18-OCH3 in combination with bortezomib

| | | | Cytotoxicity |
|---|---|---|---|
| Cell line | ET-18-OCH3 | Bortezomib | (%) |
| Jurkat | 2.5 μg/ml | | 2.1 ± 1.0 |
| | 5.0 μg/ml | | 13.1 ± 5.3 |
| | | 5 ng/ml | 0.4 ± 0.5 |
| | | 10 ng/ml | 3.6 ± 1.4 |
| | 2.5 μg/ml | 5 ng/ml | 11.7 ± 6.1 |
| | 2.5 μg/ml | 10 ng/ml | 18.3 ± 3.4 |
| | 5.0 μg/ml | 5 ng/ml | 24.1 ± 10.0 |
| | 5.0 μg/ml | 10 ng/ml | 37.5 ± 7.4 |
| HL60 | 2.5 μg/ml | | 11.3 ± 7.3 |
| | 5.0 μg/ml | | 35.0 ± 15.9 |
| | | 5 ng/ml | 2.7 ± 1.9 |
| | | 10 ng/ml | 10.9 ± 3.7 |
| | 2.5 μg/ml | 5 ng/ml | 26.3 ± 11.0 |
| | 2.5 μg/ml | 10 ng/ml | 52.5 ± 9.2 |
| | 5.0 μg/ml | 5 ng/ml | 53.6 ± 16.7 |
| | 5.0 μg/ml | 10 ng/ml | 69.1 ± 10.4 |
| RPMI8226 | 2.5 μg/ml | | 11.2 ± 8.1 |
| | 5.0 μg/ml | | 34.7 ± 13.2 |
| | | 5 ng/ml | 7.3 ± 4.5 |
| | | 10 ng/ml | 25.1 ± 4.0 |
| | 2.5 μg/ml | 5 ng/ml | 29.7 ± 8.3 |
| | 2.5 μg/ml | 10 ng/ml | 44.4 ± 13.9 |
| | 5.0 μg/ml | 5 ng/ml | 53.6 ± 16.7 |
| | 5.0 μg/ml | 10 ng/ml | 69.1 ± 10.4 |

TABLE 2
Effect of ET-18-OCH3 in combination with fludarabine

| | | | Cytotoxicity |
|---|---|---|---|
| Cell line | ET-18-OCH3 | Fludarabine | (%) |
| Jurkat | 2.5 μg/ml | | 2.1 ± 1.0 |
| | 5.0 μg/ml | | 13.1 ± 5.3 |
| | | 10 μM | 2.4 ± 0.9 |
| | | 50 μM | 6.2 ± 2.4 |
| | 2.5 μg/ml | 10 μM | 5.1 ± 2.6 |
| | 2.5 μg/ml | 50 μM | 18.0 ± 5.0 |
| | 5.0 μg/ml | 10 μM | 25.1 ± 3.9 |
| | 5.0 μg/ml | 50 μM | 36.1 ± 9.9 |
| HL60 | 2.5 μg/ml | | 11.3 ± 7.3 |
| | 5.0 μg/ml | | 35.0 ± 15.9 |
| | | 10 μM | 13.8 ± 9.4 |
| | | 50 μM | 37.2 ± 4.6 |
| | 2.5 μg/ml | 10 μM | 54.5 ± 8.2 |
| | 2.5 μg/ml | 50 μM | 81.0 ± 7.3 |
| | 5.0 μg/ml | 10 μM | 62.3 ± 15.6 |
| | 5.0 μg/ml | 50 μM | 84.9 ± 7.5 |

TABLE 3
Effect of ET-18-OCH3 in combination with cytarabine

| Cell line | ET-18-OCH3 | Cytarabine | Cytotoxicity (%) |
|---|---|---|---|
| Jurkat | 2.5 μg/ml | | 2.1 ± 1.0 |
| | 5.0 μg/ml | | 13.1 ± 5.3 |
| | | 10 μM | 11.1 ± 0.5 |
| | | 40 μM | 12.4 ± 1.2 |
| | 2.5 μg/ml | 10 μM | 22.7 ± 6.5 |
| | 2.5 μg/ml | 40 μM | 20.5 ± 5.5 |
| | 5.0 μg/ml | 10 μM | 39.5 ± 7.0 |
| | 5.0 μg/ml | 40 μM | 40.5 ± 11.1 |
| HL60 | 2.5 μg/ml | | 11.3 ± 7.3 |
| | 5.0 μg/ml | | 35.0 ± 15.9 |
| | | 10 μM | 24.0 ± 2.8 |
| | | 40 μM | 26.9 ± 5.0 |
| | 2.5 μg/ml | 10 μM | 66.2 ± 12.3 |
| | 2.5 μg/ml | 40 μM | 70.8 ± 15.5 |
| | 5.0 μg/ml | 10 μM | 85.0 ± 9.2 |
| | 5.0 μg/ml | 40 μM | 82.0 ± 9.6 |

TABLE 4
Effect of ET-18-OCH3 in combination with etoposide

| Cell line | ET-18-OCH3 | Etoposide | Cytotoxicity (%) |
|---|---|---|---|
| Jurkat | 2.5 μg/ml | | 2.1 ± 1.0 |
| | 5.0 μg/ml | | 13.1 ± 5.3 |
| | | 1.0 μM | 5.9 ± 1.1 |
| | | 2.5 μM | 10.0 ± 0.8 |
| | | 5.0 μM | 16.8 ± 8.4 |
| | 2.5 μg/ml | 1.0 μM | 10.1 ± 2.3 |
| | 2.5 μg/ml | 2.5 μM | 20.8 ± 3.3 |
| | 2.5 μg/ml | 5.0 μM | 26.0 ± 6.6 |
| | 5.0 μg/ml | 1.0 μM | 33.4 ± 11.0 |
| | 5.0 μg/ml | 2.5 μM | 40.3 ± 13.2 |
| | 5.0 μg/ml | 5.0 μM | 37.6 ± 8.5 |
| HL60 | 2.5 μg/ml | | 11.3 ± 7.3 |
| | 5.0 μg/ml | | 35.0 ± 15.9 |
| | | 1.0 μM | 4.1 ± 0.7 |
| | | 2.5 μM | 10.7 ± 5.4 |
| | | 5.0 μM | 23.3 ± 8.2 |
| | 2.5 μg/ml | 1.0 μM | 22.0 ± 11.4 |
| | 2.5 μg/ml | 2.5 μM | 58.0 ± 18.3 |
| | 2.5 μg/ml | 5.0 μM | 74.1 ± 15.3 |
| | 5.0 μg/ml | 1.0 μM | 59.2 ± 19.8 |
| | 5.0 μg/ml | 2.5 μM | 81.1 ± 8.2 |
| | 5.0 μg/ml | 5.0 μM | 85.6 ± 7.7 |

From the above results it is apparent that an adjunction of 1-O-octadecyl-2-β-methyl-glycero-3-phosphocholine (ET-18-OCH3) with any of the anti-neoplastic agents tested was giving rise to an improved cytotoxic effect in either of the cell limes employed. Importantly, the different combinations of active ingredients acted not only in an additive but in a synergistic manner. Thus, this synergistic mode of action allows for the administration of reduced drug doses in order to achieve the same pharmaceutical efficacy as compared to an individual administration, which will again reduce the risk of the occurrence of adverse side effects.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

All documents cited or referenced herein including any manufacturer's instructions, descriptions, product specifications, and product sheets for any products mentioned herein or in any document referenced herein, are hereby incorporated by reference, and may be employed in the practice of the invention. Citation or identification of any document in this application is not an admission that such document is available as prior art to the present invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-29. (canceled)

30. Use of a tri-substituted glycerol compound according to formula (I) 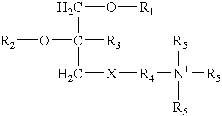
or an enantiomer or diastereomer or a pharmaceutically acceptable salt thereof and at least one pharmaceutically acceptable excipient for the manufacture of a medicament for the treatment of hematological malignancies, wherein
X is selected from the group consisting of phosphate and sulfate;
R₁ is selected from the group consisting of C₁₆-C₂₀ alkyl;
R₂ is selected from the group consisting of C₁-C₃ alkyl and C₁-C₃ hydroxyalkyl;
R₃ is selected from the group consisting of hydrogen and C₁-C₃ alkyl;
R₄ is selected from the group consisting of C₁-C₃ alkyl and C₃-C₆ cycloalkyl; and
R₅ is selected from the group consisting of hydrogen and methyl.

31. The use according to claim 30, wherein X is phosphate, R₁ is —(CH₂)₁₇—CH₃, R₂ is CH₃, R₃ is H, R₄ is —(CH₂)₂—, and R₅ is CH₃.

32. The use according to claim 30, wherein the medicament is a pharmaceutical solid dosage form for oral administration, preferably an enteric dosage form.

33. The use according to claim 32, wherein the pharmaceutical solid dosage form is selected from the group consisting of tablets, pills, capsules, and granules.

34. The use according to claim 30, wherein the hematological malignancies are selected from the group consisting of leukemia, lymphoma, multiple myeloma, and myelodysplastic syndrome, with the leukemia preferably being selected from the group consisting of acute myeloid leukemia, acute lymphoid leukemia, chronic myeloid leukemia, and chronic lymphoid leukemia; or with the lymphoma preferably being selected from the group consisting of Hodgkin lymphoma and non-Hodgkin lymphoma.

35. The use according to claim 30, wherein the medicament is used in combination with at least one other pharmaceutical comprising one or more additional active ingredients.

36. The use according to claim 35, wherein the one or more additional active ingredients are selected from the group consisting of anti-neoplastic agents.

37. The use according to claim 36, wherein the anti-neoplastic agents are selected from the group consisting of alkylating agents, antimetabolites, plant alkaloids, antibiotics, inhibitors of topoisomerases I and/or II, steroids, inhibitors of tyrosine kinases, proteosome inhibitors, and DNA intercalating agents, with the alkylating agents preferably being selected from the group of nitrogen mustards, nitrosoureas, platinum derivatives, and alkylsulfonates; or with the antimetabolites preferably being selected from the group consisting of methotrexate, purine analogs, and pyrimidine analogs.

38. The use according to claim 36, wherein the anti-neoplastic agents are selected from the group consisting of chlorambucil, cyclophosphamide, melphalan, mechlorethamine, carmustine, cisplatin, carboplatin, busulfan, treosulfan, methotrexate, fludarabine, clofarabine, pentostatine, cladribine, cytarabine, decitabine, vincristine, vinblastine, mitoxantrone, epirubicin, doxorubicin, daunorubicin, bleomycin, etoposide, teniposide, dexamethasone, imatinib, bortezomib, amsacrine, and thalidomide.

39. The use according to claim 35, wherein the one or more additional active ingredients are selected from the group of antibodies, wherein said antibodies are directed against one or more epitopes on the cell surface of hematological cells.

40. Tri-substituted glycerol compound as defined in claim 30 for the treatment of hematological malignancies, wherein the hematological malignancies are preferably selected from the group consisting of leukemia, lymphoma, multiple myeloma, and myelodysplastic syndrome.

41. Method for the treatment of hematological malignancies, comprising:
(a) administering to a patient a medicament or a combination of medicaments as defined in claim 30; and optionally
(b) determining the extent of Fas receptor gene expression in one or more malignant cells of the patient to be treated before administering the medicament or the combination of medicaments.

42. The method according to claim 41, further comprising:
(c) determining one or more of the following in the one or more tumor cells of the patient to be treated before administering the medicament or the combination of medicaments:
(i) the presence or absence of one or more ras gene mutations;
(ii) the presence or absence of one or more FGFR3 gene mutations; and
(iii) the presence or absence of one or more PTEN gene mutations,
wherein the presence or absence of the one or more FGFR3 gene mutations is preferably determined in the concomitant presence of the t(4;14) chromosomal translocation.

43. The method according to claim 41 or 42, further comprising:
(d) comparing the results of the measurements obtained in (b) and/or (c) with those obtained in one or more control cells.

44. In vitro method for determining the susceptibility of one or more malignant cells to a medicament or a combination of medicaments as defined in claim 30, the method comprising:
(a) determining the extent of Fas receptor gene expression in the one or more malignant cells.

45. The in vitro method according to claim 44, further comprising:
(b) determining one or more of the following in the one or more malignant cells:
(i) the presence or absence of one or more ras gene mutations;
(ii) the presence or absence of one or more FGFR3 gene mutations; and
(iii) the presence or absence of one or more PTEN gene mutations,
wherein the presence or absence of the one or more FGFR3 gene mutations is preferably determined in the concomitant presence of the t(4;14) chromosomal translocation.

46. The in vitro method according to claim 44 or 45, further comprising:
(c) comparing the results of the measurements obtained in (a) and/or (b) with those obtained in one or more control cells.

47. Kit-of-parts, comprising a medicament as defined in claim 30.

48. The kit-of-parts according to claim 47, further comprising a medicament as defined in claim 35.

49. Kit-of-parts, comprising a tri-substituted glycerol compound as defined in claim 40.
